(19) United States
(12) Patent Application Publication
(10) Pub. No.: US 2013/0158513 A1
(43) Pub. Date: Jun. 20, 2013

(54) NOZZLE APPARATUS AND METHODS OF USING SAME

(71) Applicants: Jay Betz, Tulsa, OK (US); Daniel Darst, Zimmerman, MN (US); William Robert Worrell, Hopkins, MN (US); Eric Jason Krause, Big Lake, MN (US)

(72) Inventors: Jay Betz, Tulsa, OK (US); Daniel Darst, Zimmerman, MN (US); William Robert Worrell, Hopkins, MN (US); Eric Jason Krause, Big Lake, MN (US)

(21) Appl. No.: 13/525,019

(22) Filed: Jun. 15, 2012

Related U.S. Application Data
(60) Provisional application No. 61/497,355, filed on Jun. 15, 2011.

(51) Int. Cl. A61M 3/02

(52) U.S. Cl. CPC A61M 3/0279
USPC 604/514; 604/275; 604/257

(57) ABSTRACT

In accordance with some embodiments, a nozzle apparatus includes a body having a first portion and a second portion. The first portion has a gripping portion for attaching and removing the nozzle apparatus from a container. In one embodiment, the second portion is sized and configured to be positioned in at least one nostril of an individual. A portion of the second portion being asymmetrical to the first portion of the body. The first and second portions of the body define a fluid passageway between an inlet and an outlet of the body. A portion of the fluid passageway is centrally disposed from the inlet of the body along an axis of the body. A portion of the fluid passageway angles obliquely substantially near the outlet of the body. The outlet of the fluid passageway has a diameter greater than the diameter of the passageway.

### CROSS-REFERENCE TO RELATED APPLICATIONS

The present application claims the benefit under 35 U.S.C. §119(e) of U.S. Provisional application Ser. No. 61/497,355, filed Jun. 15, 2011, entitled “Nozzle Apparatus and Methods of Using Same”, which is hereby expressly incorporated herein in its entirety.

### STATEMENT REGARDING FEDERALLY SPONSORED RESEARCH OR DEVELOPMENT

Not applicable.

### FIELD OF THE INVENTION

The present invention relates generally to a nozzle apparatus for rinsing nasal passages, and more particularly, but not by way of limitation, to an improved nozzle apparatus for rinsing nasal passages and a method of using the nozzle apparatus.

### BACKGROUND OF THE INVENTION

A nasal passage extends from the nostrils of the nose to the pharynx and is lined with mucosa. There are three turbinates (inferior, middle, superior) in each nasal passage. Each one protrudes from the sidewall of the nose. Turbinates divide the nasal passage into four groove-like air passages. Turbinates increase the inner surface area of the nose and act to warm, humidify and filter air before it passes on to the throat. The inferior turbinates are responsible for the majority of airflow direction. Most inhaled airflow travels between the inferior turbinate and the middle turbinate.

Sinusitis is an inflammation of the mucosa of various sinuses, or spaces, which are located around the nasal passages. Rhinitis is an inflammation of the mucosa of the nasal passages. Sinusitis and rhinitis can be caused by cold viruses, allergies to various allergens, smoking, bacterial or fungal infections, nasal polyps, deviated nasal septums and non-allergic hypersensitivities.

Nasal rinsing is beneficial in the treatment and prevention of allergic rhinitis and sinus infections. During nasal rinsing or washing, the nasal passages are washed to flush out excess mucous and debris from the nose and sinuses and/or turbinates. Rinsing helps to prevent the crusting of secretions in the nasal passages which can block sinuses from draining. The blockage of the drainage sites could also occur with swelling from allergies or irritants which could result in infection. The rinsing helps to reduce tissue swelling in the nasal passages, which also results in the clearance of mucous. Typically, a saline solution is used to rinse the nasal passages; however, it should be understood by one of ordinary skill in the art that various solutions may be utilized for nasal rinsing.

To this end, although apparatuses of the existing art are operable for rinsing nasal passages, further improvements are desirable and a need remains to provide a nozzle apparatus and method of use whereby the rinsing of the nasal passages of all sizes is enhanced. It is to such a nozzle apparatus and method of making and using that at least one embodiment of the present invention is directed.

### SUMMARY

In accordance with embodiments disclosed herein, a nozzle apparatus includes a body having a first portion and a second portion. The first portion has a gripping portion for attaching and removing the nozzle apparatus from a container. The second portion is sized and configured to be positioned in at least one nostril of an individual. The first portion and second portion define a fluid passageway between an inlet and an outlet of the body. A portion of the second portion is asymmetrical to the first portion of the body. A portion of the fluid passageway is centrally disposed from the inlet of the body along an axis of the body. A portion of the fluid passageway angles obliquely substantially near the outlet of the body. The outlet of the fluid passageway has a diameter greater than the diameter of the passageway. In another embodiment of the present invention, a nozzle apparatus assembly includes the nozzle apparatus having a body as described herein, and a container. The container has a fluid and is removably attached to the body. The container is in fluid communication with the body.

In another embodiment, a method of dispersing a fluid into a nasal passageway is disclosed. A nozzle apparatus having a body as described herein, and a container as described herein, are provided. The container is removably attached to a portion of the body of the nozzle apparatus. A gripping portion is provided to the first portion of the body of the nozzle apparatus. The container is attached to the body of the nozzle apparatus. The container may be removed from the body of the nozzle apparatus. The outlet of the body is positioned in a nasal passageway of an individual. The fluid is delivered to the nasal passageway of the individual. The outlet of the body of the nozzle apparatus is moved between a first position and a second position. In the first position, the outlet of the body of the nozzle apparatus is positioned in one nostril of the individual and in the second position, the outlet of the nozzle apparatus is positioned in the opposing nostril of the individual. These and other features and advantages which may characterize various embodiments can be understood in view of the following detailed discussion and the accompanying drawings.

### BRIEF DESCRIPTION OF THE SEVERAL VIEWS OF THE DRAWINGS

FIG. 1 is a top perspective view of one embodiment of a nozzle apparatus constructed in accordance with the present invention.

FIG. 2 is a top view of the nozzle apparatus of FIG. 1.

FIG. 3 is a cross-sectional view of the nozzle apparatus of FIG. 1 shown removably attached to a container.

FIG. 4 is an elevation view of the nozzle apparatus of FIG. 1.

FIG. 5 is a posterior view of the nozzle apparatus of FIG. 1.

FIG. 6 is a side view of the nozzle apparatus of FIG. 1 in the first position.

FIG. 7 is a side view of the nozzle apparatus of FIG. 1 in the second position.

### DETAILED DESCRIPTION OF THE DRAWINGS

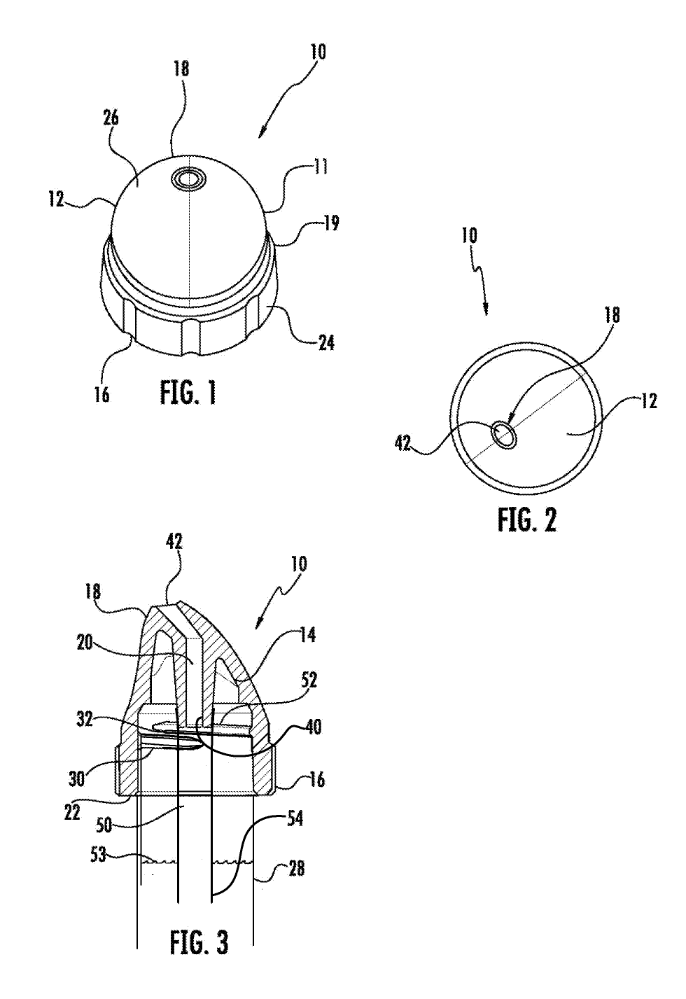

Referring now to the drawings, and more particularly to FIGS. 1-7, shown therein is one embodiment of a nozzle apparatus 10 constructed in accordance with the present invention for rinsing nasal passages. The nozzle apparatus 10 has a body 11 which includes an exterior 12, an interior 14, a first end 16, a second end 18, a sidewall 19, and a passageway 20 extending between the first end 16 and the second end 18.

It is contemplated, in one embodiment, that the nozzle apparatus 10 is constructed from a single piece of material, however, it should be understood by one of ordinary skill in the art that the nozzle apparatus 10 may be constructed from various components. The nozzle apparatus 10 is preferably made of durable materials which are strong enough to allow for use of the nozzle apparatus 10. The nozzle apparatus 10 may be constructed from a variety of durable and resilient materials, such as for example, polymeric materials, plastics, thermoplastics, elastomers, rubber, cardboard, metals, such as aluminum, steel, titanium, magnesium or alloys containing these metals, and composite materials which are capable of providing the desired strength and durability for the nozzle apparatus 10. Further, it should be understood that the nozzle apparatus 10 may be constructed from materials that may be water-resistant, anti-microbial, or waterproof, so that the nozzle apparatus 10 may be easily cleaned and/or disinfected.

The exterior 12 of the body 11 of the nozzle apparatus 10 has a first portion 24 and a second portion 26. In one embodiment, the first portion 24 of the exterior 12 of the body 11 is substantially circular, and the second portion 26 of the exterior 12 of the body 11 is generally asymmetrically shaped. The first portion 24 may include ridges on a portion of the surface of the exterior 12 of the body 11 to provide a gripping portion for attaching and removing the nozzle apparatus 10 from a container. A portion of the second portion 26 is sized and configured so as to be positioned in at least one nostril of an individual. It should be understood that as long as a generally asymmetrical shape is maintained for the second portion 26 of the nozzle apparatus 10, any shape, whether geometric, or non-geometric, may be utilized for the nozzle apparatus 10.

The first end 16 of the nozzle apparatus 10 has an opening 22 which is sized and configured to removably attach to a container 28 having an opening 32. The container 28 typically is constructed of a resilient material, the type typically used in nasal rinsing, so as to enable an individual to squeeze the container 28 and thus, provide an internal pressure suitable to force a directed stream of fluid out of the container 28 into the nasal passages. It should be understood that other dispensing methods can be utilized. In one embodiment, the container 28 may be a standard high-density polyethylene (HDPE) or low-density polyethylene (LDPE) bottle (i.e., a bottle with a 24-410 neck finish (24 millimeter diameter and 1 and ½ thread turns) which may hold a fluid, such as saline solution, for rinsing nasal passages. It should be understood by one of ordinary skill in the art that any type of material (such as polyethylene or polypropylene), shape, or size of container 28 may be utilized with the nozzle apparatus 10, so long as the container 28 functions in accordance with the present invention. Further, it should be understood by one of ordinary skill in the art that any fluid may be utilized herein, so long as the fluid functions in accordance with the present invention as described herein.

The interior 14 is provided with a threaded portion 30 substantially near the first end 16 for attaching the nozzle apparatus 10 to the container 28 so as to maintain a liquid-tight seal. It should be understood by one of ordinary skill in the art that any method of removably attaching one object to another may be utilized, such as a snap-fit attachment, exterior clamping, or the like, so long as the attachment method functions in accordance with the present invention as described herein.

The passageway 20 of the nozzle apparatus 10 extends between an inlet 40, substantially near the first end 16 of the nozzle apparatus 10, and an outlet 42 of the second end 18 of the nozzle apparatus 10. A portion of the passageway 20 is centrally disposed from the first end 16 along the axis of the body 11 of the nozzle apparatus 10 substantially as shown, and angles obliquely substantially near the second end 18 of the nozzle apparatus 10. The oblique portion of the passageway 20 creates the appropriate turbulence needed to enhance the wash effect of the fluid on the nasal passages when nasal rinsing. It should be understood that the passageway 20 may be configured to angle at various places along the length of the body 11 of the nozzle apparatus 10. The outlet 42 of the passageway 20 of the nozzle apparatus 10 has a diameter 44 greater than the diameter of the passageway 20 for various uses. For example, when nasal rinsing, the greater diameter 44 of the outlet 42 of the passageway 20 allows for the flow of fluid from the outlet 42 to be dispersed to achieve a broader coverage in the nasal passages of fluid and slightly reduces the velocity of the fluid which improves the comfort of the nasal washing.

The design of the nozzle apparatus 10 and the directed flow it creates enhances the effectiveness of nasal washing by properly channeling the flow across all three nasal turbinates.

FIG. 3 shows the nozzle apparatus 10 utilized with the container 28 for dispensing a fluid 53. In one embodiment, a conventional tube 50 is sized and configured so that one end 52 of the tube 50 is removably attached to a portion of the inlet 40 of the passageway 20 so that the passageway 20 of the nozzle apparatus 10 is in fluid communication with the container 28. The tube 50 may be removably attached in a variety of ways. The tube 50 extends into the container 28 a desired distance, depending on the size and shape of the container 28, so that another end 54 of the tube 50 communicates with the fluid 53 in the container 28. Proper flow of the fluid 53 from the container 28 to the passageway 20 of the nozzle apparatus 10 is maintained. For example, in one embodiment, the tube 50 may extend into the container 28 such that the end 54 is approximately ⅛ to 3/16 inches from the bottom of the container 28.

Additionally, the nozzle apparatus 10 is designed so that it can be used without the tube 50 while still achieving proper flow characteristics and comfort of use. With the tube 50 removed, the opening 22 of the first end 16 of the nozzle apparatus 10 is in fluid communication with the opening 32 of the container 28. Once the container 28 is properly filled, the nozzle apparatus 10 may be inserted into the nostril as previously described, but by turning the head at a roughly 45° angle opposite the nozzle apparatus 10, the nozzle apparatus 10 may be used in a manner similar to a neti-pot or other gravity flow devices.

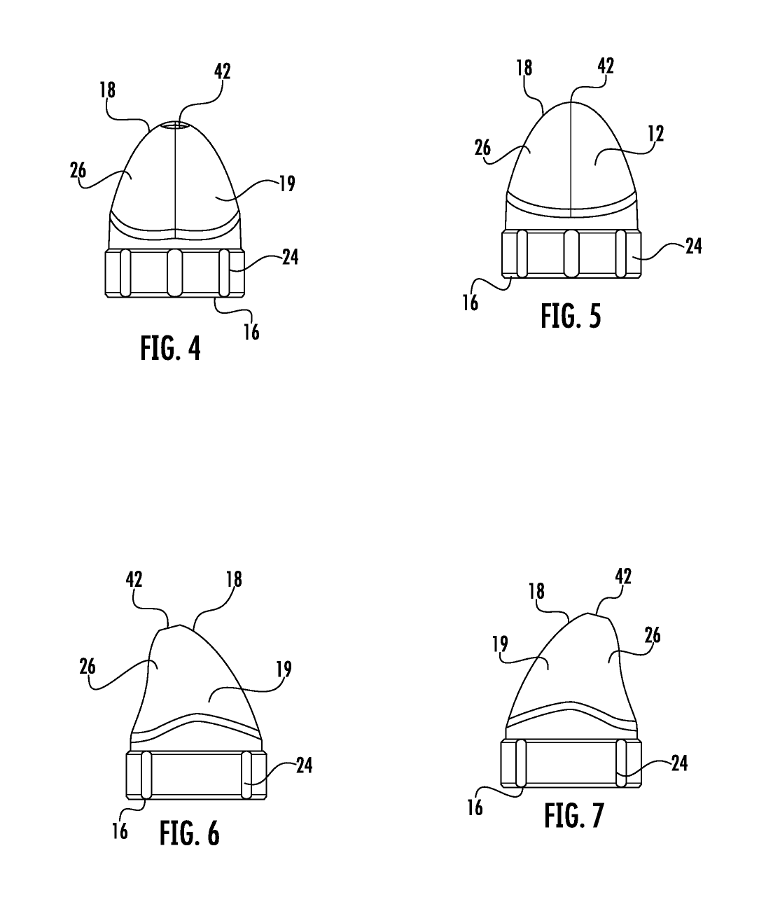

Referring to FIGS. 6 and 7, the nozzle apparatus 10 is movable between a first position (FIG. 6) and a second position (FIG. 7). In the first position, the second end 18 of the nozzle apparatus 10 is manually movable such that the outlet 42 is oriented toward the outward portion of the left nostril to be positioned in the left nostril of an individual. An “L” may be printed or imprinted on the exterior 12 of the body 11 of the nozzle apparatus 10 to indicate the proper first position to the individual. In the second position, the second end 18 of the nozzle apparatus 10 is manually movable such that the outlet 42 is oriented toward the outward portion of the right nostril to be positioned in the right nostril of the individual. An “R” may be printed or imprinted on the exterior 12 of the body 11 of the nozzle apparatus 10 to indicate the proper second position to the individual. The self-orienting design properly positions the outlet 42 of the nozzle apparatus 10 into the proper nostril so that the flow of fluid is directed at the inferior, middle, and superior turbinates to flush these key nasal structures that retain molds and allergens that can trigger allergic reactions and accompanying complications.

In use, an individual moves the nozzle apparatus 10 into the first position and places the outlet 42 of the nozzle apparatus 10 into the left nostril. The container 28 is squeezed by a hand of the individual to force fluid out of the container 28, through the nozzle apparatus 10, and into the left nasal passageway until the desired rinsing of the left nasal passageway is complete. The individual next moves the nozzle apparatus 10 into the second position and places the outlet 42 of the nozzle apparatus 10 into the right nostril. The container 28 is squeezed by a hand of the individual to force fluid out of the container 28, through the nozzle apparatus 10, and into the right nasal passageway until the desired rinsing of the right nasal passageway is complete.

While one embodiment of the present invention is described herein utilized in a nasal application, it should be understood by one of ordinary skill in the art, the apparatus and methods of the present invention may be readily applied to use in other applications, such as oral, optic, anal, and the like. It should be understood that changes may be made in the operation and the setup of the present invention accordingly.

From the above description, it is clear that the present invention is well adapted to carry out the objects and to attain the advantages mentioned herein as well as those inherent in the invention. While a presently preferred embodiment of the invention has been described for purposes of this disclosure, it will be understood that numerous changes may be made which will readily suggest themselves to those skilled in the art and which are accomplished within the spirit of the invention disclosed.

What is claimed is:

1. A nozzle apparatus, comprising:
a body having a first portion and a second portion defining a fluid passageway between an inlet and an outlet of the body, a portion of the second portion being asymmetrical to the first portion of the body.

2. The nozzle apparatus of claim 1, wherein the first portion has a gripping portion for attaching and removing the nozzle apparatus from a container.

3. The nozzle apparatus of claim 1, wherein the second portion is sized and configured to be positioned in at least one nostril of an individual.

4. The nozzle apparatus of claim 1 wherein a portion of the fluid passageway is centrally disposed from the inlet of the body along an axis of the body.

5. The nozzle apparatus of claim 1 wherein a portion of the fluid passageway angles obliquely substantially near the outlet of the body.

6. The nozzle apparatus of claim 1 wherein the outlet of the fluid passageway has a diameter greater than the diameter of the passageway.

7. A nozzle apparatus assembly, comprising:
a body having a first portion and a second portion defining a fluid passageway between an inlet and an outlet of the body, a portion of the second portion being asymmetrical to the first portion of the body; and
a container having a fluid, the container removably attached to the body wherein the container is in fluid communication with the body.

8. The assembly of claim 7, wherein the first portion of the body has a gripping portion for attaching and removing the body from the container.

9. The assembly of claim 7, wherein the second portion is sized and configured to be positioned in at least one nostril of an individual.

10. The assembly of claim 7 wherein a portion of the fluid passageway is centrally disposed from the inlet of the body along an axis of the body.

11. The assembly of claim 7 wherein a portion of the fluid passageway angles obliquely substantially near the outlet of the body.

12. The assembly of claim 7 wherein the outlet of the fluid passageway has a diameter greater than the diameter of the passageway.

13. A method of dispersing a fluid into a nasal passageway, comprising the steps of:
providing a nozzle apparatus having a body having a first portion and a second portion defining a fluid passageway between an inlet and an outlet of the body, a portion of the second portion being asymmetrical to the first portion of the body;
providing a container having a fluid, the container removably attached to a portion of the body of the nozzle apparatus;
positioning the outlet of the body in a nasal passageway of an individual; and
delivering the fluid to the nasal passageway of the individual.

14. The method of claim 13, further comprising the step of:
moving the outlet of the body of the nozzle apparatus between a first position and a second position.

15. The method of claim 13 wherein in the first position, the outlet of the body of the nozzle apparatus is positioned in one nostril of the individual and in the second position, the outlet of the nozzle apparatus is positioned in the opposing nostril of the individual.

16. The method of claim 13, further comprising:
providing a gripping portion to the first portion of the body of the nozzle apparatus;
attaching the container to the body of the nozzle apparatus; and
removing the container from the body of the nozzle apparatus.

17. The method of claim 13, wherein the second portion is sized and configured to be positioned in at least one nostril of an individual.

18. The method of claim 13 wherein a portion of the fluid passageway is centrally disposed from the inlet of the body along an axis of the body.

19. The method of claim 13 wherein a portion of the fluid passageway angles obliquely substantially near the outlet of the body.

20. The method of claim 13 wherein the outlet of the fluid passageway has a diameter greater than the diameter of the passageway.
